(19) United States
(12) Patent Application Publication
(10) Pub. No.: US 2016/0151626 A1
(43) Pub. Date: Jun. 2, 2016

(54) Stimulation and Electromyography Detection

(71) Applicant: ANALYTICA LIMITED, Brisbane (AU)

(72) Inventors: Michael Maurice Gorman, Carnegie (AU); Thomas Aidan Stamp, Woodend (AU); Peter Aubrey Bartlett, Algester (AU)

(21) Appl. No.: 14/903,815

(22) PCT Filed: Jun. 17, 2014
(86) PCT No.: PCT/AU2014/000622

§ 371 Date: Jan. 8, 2016

(30) Foreign Application Priority Data
Jul. 11, 2013 (AU) 2013902557

(51) Int. Cl. A61N 1/36; A61B 5/00; A61N 1/372; A61B 5/0488

(52) U.S. Cl. CPC A61N 1/36003; A61B 5/0488; A61B 5/4836; A61N 1/37247

(57) ABSTRACT

An electronic device for treating neuromuscular disorders. The electronic device includes a current source configured to generate one or more electrical stimuli based on one or more selected parameters associated with the one or more electrical stimuli and an electromyographic (EMG) processing unit configured to process a received EMG signal. The electronic device further includes a switching unit operatively coupled to the current source and the EMG processing unit. The switching unit is configured to switch between operation of the current source and the EMG processing unit based on a selected operation mode.

### REFERENCE TO RELATED PATENT APPLICATION(S)

This application claims the benefit of the filing date of Australian Provisional Patent Application No. 2013902557, filed on 11 Jul. 2013, hereby incorporated by reference in its entirety as if fully set forth herein.

### TECHNICAL FIELD

The present invention relates generally to neuro-muscular stimulation and more particularly to methods and devices for identifying muscles/nerves, and/or treating a neuro-muscular disorder through neuro-muscular stimulation and electromyography.

### BACKGROUND

Muscular disorders such as dystrophy, polymyositis, hypertonia or spasticity affect hundreds of thousands of people each year. Generally these disorders develop in patients with cerebral palsy, a traumatic brain or spinal cord injury, stroke, or multiple sclerosis. Such muscle disorders are brought about due to an imbalance of signals from the central nervous system to the affected muscles. Muscle spasticity, for instance, is a muscle control disorder that is characterized by tight or stiff muscles and an inability to control these muscles. Additionally, in spasticity, reflexes may be very strong or may persist for extended periods of time, and involuntary movements such as spasms or clonus may occur. Furthermore, the patient may feel pain and have an abnormal posture.

Various procedures are available to treat spasticity. Physiotherapy and rehabilitation have shown promise for spastic muscles with mild-moderate impairment. For more severe spasticity, medications such as Baclofen™, Tizanidine™, Diazepam™, or Clonazepam™ have proven effective in dampening the signals between the central nervous system and the affected muscle. In addition to these procedures, local injections of Phenol or Botulinum toxin may be utilized to paralyse specific muscle groups in order to control spasms or clonus. Typically, an electromyography (EMG) device, a high voltage stimulator, and/or an ultrasound device are employed to guide the injections and to determine the location of specific muscles or nerves.

Motor neurons in the body transmit electrical signals to the muscles that cause the muscles to contract. Moreover, when the muscles contract, the muscles produce electrical activity. In case of spastic muscles, the electrical activity may be unusual. For instance, the electrical activity may be sporadic, excessive or prolonged. To identify spastic muscles, physicians may place electrodes connected to the EMG device in contact with the suspicious muscles. Subsequently, the electrical activity produced by the muscles may be received and recorded by the EMG device. A physician may interpret the EMG signals to determine whether any nerve, muscle, or nerve-to-muscle dysfunction exists. Subsequently, the physician may inject a dose of phenol or botulinum toxin to the identified site in order to paralyse the dysfunctional muscle.

Another technique often utilized by physicians to identify dysfunctional muscle is high voltage (HV) electrical stimulation. In this technology, very high voltage pulses are delivered to a muscle or group of muscles through one or more needle and/or surface electrodes. Further, the pulses act on a large part of the muscle and thus on thousands of motor units. The delivery of electrical pulses forces the patient's extremities (i.e., fingers, toes, arms, etc.) that are associated with the stimulated muscle to twitch. Based on the intensity and/or direction of this twitch, physicians typically identify the muscle into which the needle is inserted. If the ‘wrong’ extremities twitch then the needle electrode is repositioned until the correct ‘twitch’ and thus the correct muscle location is identified. Once the muscle location is identified, a dose of the paralysing agent may be injected. However, often, in large muscle groups, EMG or electrical stimulation may not be able to accurately identify the best injection site.

To improve the accuracy of identifying the correct injection site, various devices have been available that include both the stimulation and the EMG functionality. Typically, with these devices, a physician receives information about the muscle groups from both the stimulator and the EMG and then interpolates this information to determine the most accurate injection site. For instance, a physician may transmit pulses to a particular muscle group for a few seconds to identify the muscle group and then record the electrical activity of that muscle group for the next few seconds. Based on a reaction of the muscle to the stimulation and a reading of the electrical activity of the muscle, the physician may determine whether the correct muscle is identified.

These conventional devices typically include a trigger for manually switching between the stimulation and EMG functionalities. Because operation between the stimulation and EMG functionality is switched manually, the movement of the physician's hand or the distraction from the muscle site may cause the electrode to move, thereby compromising location and/or timing accuracy. Accordingly, conventional devices occasionally fail to accurately identify the best injection site and because of such inaccuracy, the physician may incorrectly inject the paralysing agent in the wrong muscle or muscle group.

### SUMMARY

It is an object of the present invention to substantially overcome, or at least ameliorate, one or more disadvantages of existing arrangements.

According to one aspect of the present disclosure, there is provided an electronic device. The electronic device includes a current source configured to generate one or more electrical stimuli based on a predetermined parameter associated with the electrical stimulus. Further, the electronic device includes an electromyographic processing unit configured to process EMG signals generated by muscles. In addition, the electronic device includes a switching unit operatively coupled to the electric source and the electromyographic processing unit. The switching unit is configured to switch between operation of the current source and the EMG processing unit based on a selected operation mode.

According to one aspect of the present disclosure, there is provided an electronic device for treating neuromuscular disorders. The electronic device includes a current source configured to generate one or more electrical stimuli based on one or more selected parameters associated with the one or more electrical stimuli and an electromyographic (EMG) processing unit configured to process a received EMG signal. The electronic device further includes a switching unit operatively coupled to the current source and the EMG processing unit. The switching unit is configured to switch between operation of the current source and the EMG processing unit based on a selected operation mode.

According to another aspect of the present disclosure, there is provided an electronic device for treating a neuro-muscular condition. The electronic device includes a current source configured to generate one or more electrical stimuli based on a predetermined parameter, and an electromyographic (EMG) processing unit configured to process an EMG signal. Further, the electronic device includes a switching unit operatively coupled to the current source and the EMG processing unit in a time-domain multiplexed manner. The switching unit is configured to deliver the one or more electrical stimuli from the current source to a patient and transmit the EMG signal from the patient to the EMG processing unit, substantially simultaneously. Moreover, the electronic device includes an output unit configured to generate an output signal corresponding to the processed EMG signal.

According to yet another aspect of the present disclosure, there is provided a method for treating a neuromuscular disorder. The method includes the steps of retrieving one or more parameters associated with electrical stimuli, and operatively coupling a current source and an EMG processing unit to a patient in a time domain multiplexed manner, where the timing of the multiplexing operation is based on the one or more retrieved parameters of the electrical stimuli. Further, the method includes the steps of delivering one or more electrical stimuli from the current source to a patient when the current source is operatively coupled to the patient, receiving an EMG signal from the patient when the EMG processing unit is operatively coupled to the patient, and processing the received EMG signal to generate an output signal.

### BRIEF DESCRIPTION OF THE DRAWINGS

FIG. 1 is a schematic block diagram illustrating an environment where an electronic device according to the present disclosure may be applied, in one example;

FIGS. 2A and 2B collectively form a schematic block diagram representation of the electronic device of FIG. 1 upon which described arrangements can be practised;

FIG. 3 is a schematic block diagram of the electronic device of FIGS. 1-2;

FIGS, 4A, 4B, 4C and 4D show graphical representations illustrating various operation modes of the electronic device; and

FIGS. 5A-5D collectively form a flowchart illustrating a method for treating a neuromuscular disorder using the electronic device of FIGS. 1-3.

While the invention is amenable to various modifications and alternative forms, specific embodiments are shown by way of example in the drawings and are described in detail. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION

Devices and methods for identifying muscles and/or nerves in a patient's body and further for treating a condition of the muscles/nerves, are described below. More particularly, the methods and devices are configured to identify muscles/nerves and their condition based on an evaluation of electric activity produced by the patient's body and/or the contraction of a patient's muscles due to electrical stimulation. To this end, the device includes a stimulator configured to deliver electrical stimuli to a specific site in a patient's body. The electrical stimuli cause specific muscles and/or nerves to “twitch” or contract. Moreover, as referred to herein, electrical stimuli may be in the form of constant current, short duration, direct current (DC) electrical signals and the like, including sinusoidal signals, square-wave signals, exponential signals or linear signals. Furthermore, the electrical stimuli may be high voltage electrical signals that are able to produce a detectable response in the muscles/nerves.

The device further includes an EMG processing unit configured to receive and record EMG signals produced by the patient's muscles. The EMG processing unit is also configured to process the EMG signals and generate an output signal. In addition, the device includes a switching unit that is configured to automatically switch between operation of the stimulator and the EMG processing unit based on a selected operation mode.

In one arrangement, the device may be configured to substantially simultaneously perform stimulation and EMG evaluation. Such a simultaneous operation is possible because the duty cycle of the electrical stimulus is low. For instance, in case that the stimulator transmits a stimulus every 200 ms and the duration of the stimulus is 1 ms, the time period between stimuli (i.e., 199 ms) is not utilized. The described arrangements utilize this unused time period between electrical stimuli to receive and record the EMG signals. Accordingly, the device simultaneously and seamlessly transmits electrical stimuli to the patient and evaluates the EMG signals produced by the patient's muscles during the interval between electrical stimuli.

Arrangements of the present disclosure are described with respect to a device utilized for injecting paralysing agents to a specific site within a patient's body. The agent may be phenol, botulinum toxin or any other such agent that paralyses muscles. Moreover, the device may be utilized to treat muscular disorders such as spasticity. A physician may also utilize the device in diagnosing neuro-muscular disorders such as dystrophy, polymyositis, or hypertonia without departing from the scope of the present disclosure. It will be understood that in diagnosing neuro-muscular disorders, the physician will typically not utilize the device to inject any agents into the patient's muscles. Instead, the device may be solely utilized to record and evaluate the EMG signal generated by the patient's muscles and/or record muscle twitching in response to the electrical stimuli.

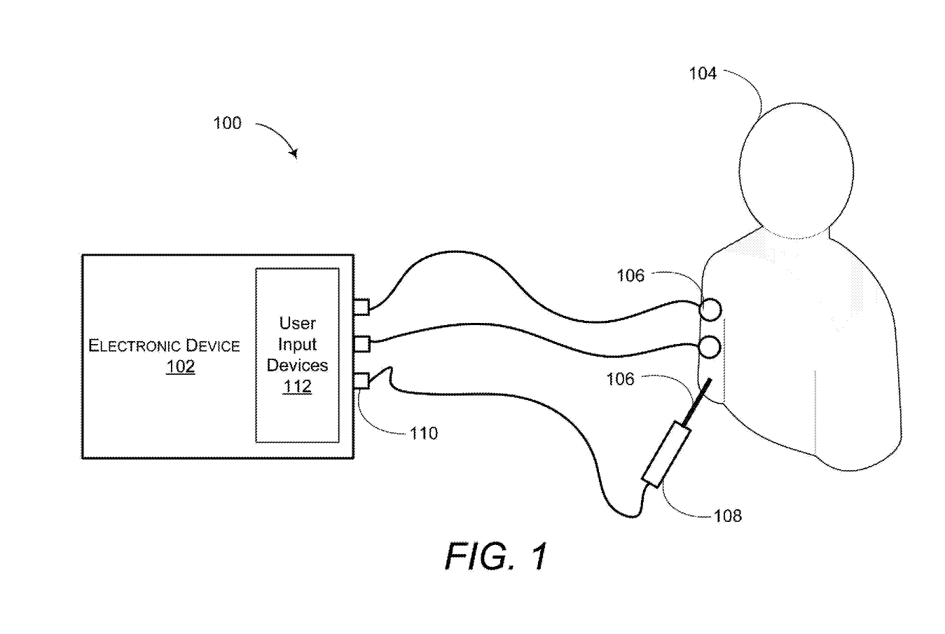

FIG. 1 is a block diagram 100 illustrating an electronic device 102 for treating a neuro-muscular disorder. The electronic device 102 includes a means for stimulation and a means for receiving and storing EMG signals. Further, the electronic device 102 may be coupled to a patient 104 by way of one or more electrodes 106. To this end, the electronic device 102 includes one or more ports 110. One end of the electrodes 106 may be electrically coupled to the ports 110, while the other end may be attached to the patient 104.

In one example, the electrodes 106 may include a reference electrode and a ground electrode. In some cases, the muscles are stimulated through the surface. In such cases, the electrodes 106 may be attached on the patient's skin in direct proximity to the muscles to be analysed. Alternatively, percutaneous intramuscular stimulation may be required. In these instances, the electrodes 106 may be placed directly in contact with the muscle to the stimulated. It will be understood that various types of surface and intramuscular electrodes 106 are currently available and any of these electrodes may be utilized without departing from the scope of the present disclosure. For instance, discoid suffice electrodes, intramuscular electrodes, epimysial electrodes, solid filiform needles, or hollow hypodermic needles may be utilized. Further, in some arrangements, the same electrodes 106 (for instance, a surface electrode and a needle electrode) may be utilized to transmit high voltage electrical stimuli to the patient's muscles and to receive EMG signals from the patient. Alternatively, one set of electrodes 106 (such as a needle electrode and a surface electrode) may be employed for stimulation, while another set of electrodes 106 (such as a needle and a surface electrode Of both surface electrodes) may be utilized to receive EMG signals.

In the case that the electronic device 102 is utilized for injecting an agent to specific sites within a patient's body, the electrode 106, such as a needle electrode may be mounted on a syringe 108 that contains the agent as shown in FIG. 1. Accordingly, if upon stimulation, the correct injection site is located, the syringe 108 may be utilized to inject the agent at that site without removing the needle electrode 106.

The electronic device 102 may further include user input devices 112. Examples of the user input devices 112 include switches, triggers, buttons, a display, or a touchscreen. The user input devices 112 allow an operator to control the operation of the electronic device 102. For instance, using the user input devices 112, an operator may be able to select an operation mode of the device 102 or one or more control parameters associated with the electronic device 102. Furthermore, the user input devices 112 may include controls for selecting an agent dosage and for automatically injecting the selected dose, when a specific site is located.

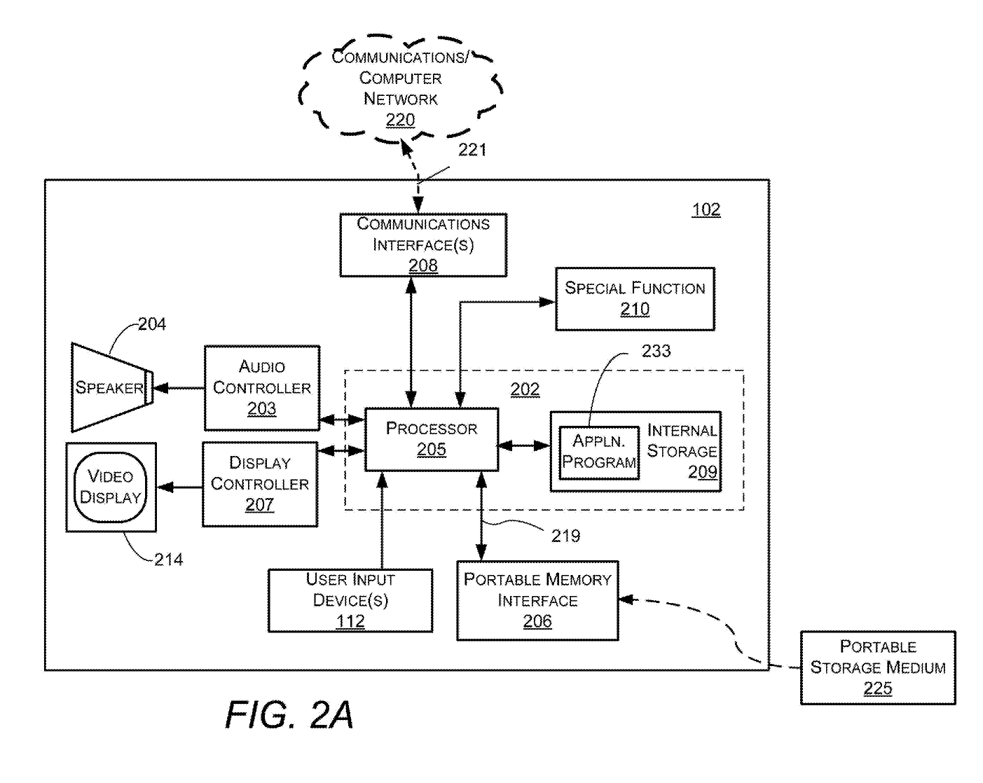

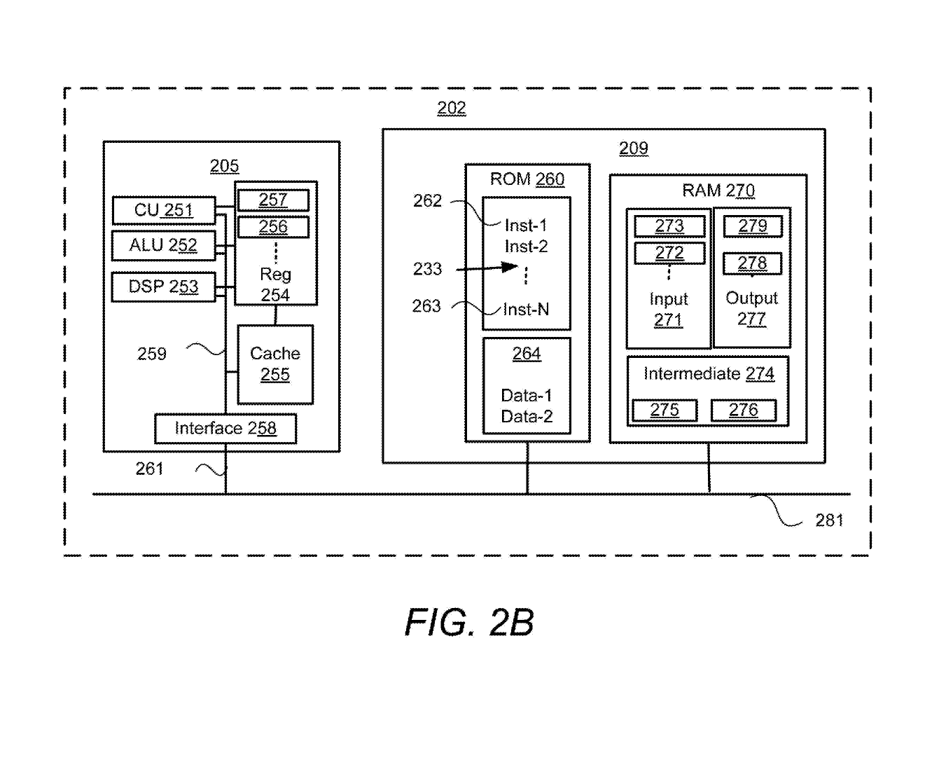

Moreover, the electronic device 102 may be a handheld device or a larger device. In the case that the electronic device 102 is configured as a handheld device, the device 102 may include one or more ergonomic features that allow an operator to conveniently carry the device in one hand and operate the user input devices 112 with fingers of the same hand. The functions of the electronic device 102 will be described in detail with reference to FIGS. 2-5.

FIGS, 2A and 2B are schematic block diagrams collectively illustrating the electronic device 102 upon which the methods of the present disclosure are desirably practiced. As described, the device 102 may include embedded components. The electronic device 102 may be used for identifying muscles/nerves, or treating neuro-muscular disorders, in which processing resources are limited. Nevertheless, the device 102 and methods of the present disclosure may also be performed on higher-level devices such as desktop computers, server computers, and other such devices with significantly larger processing resources.

As seen in FIG. 2A, the electronic device 102 includes an embedded controller 202. Accordingly, the electronic device 102 may be referred to as an “embedded device.” In the present example, the controller 202 has a processing unit 205 which is bi-directionally coupled to an internal storage module 209. The storage module 209 may be formed from non-volatile semiconductor read only memory (ROM) 260 and semiconductor random access memory (RAM) 270, as seen in FIG. 2B. The RAM 270 may be volatile, non-volatile or a combination of volatile and non-volatile memory.

The electronic device 102 further includes a display controller 207, which is connected to a video display 214, such as a liquid crystal display (LCD) panel, a CRT display or the like, and an audio controller 203, which is connected to an audio device 204, such as a speaker, headphones or the like. The display controller 207 is configured to display graphical images on the video display 214 in accordance with instructions received from the embedded controller 202, to which the display controller 207 is connected. Similarly, the audio controller 203 is configured to play audio sounds on the audio device 204 in accordance with instructions received from the embedded controller 202.

As shown in FIG. 1, the electronic device 102 also includes the user input devices 112 which are typically formed by keys, a keypad or like controls. In some implementations, the user input devices 112 may include a touch sensitive panel physically associated with the display 214 to collectively form a touch-screen. Such a touch-screen may thus operate as one form of graphical user interface (GUI) as opposed to a prompt or menu driven GUI typically used with keypad-display combinations. Other forms of user input devices may also be used, such as a microphone (not illustrated) for voice commands or a joystick/thumb wheel (not illustrated) for ease of navigation about menus.

As seen in FIG. 2A, the electronic device 102 also comprises a portable memory interface 206, which is coupled to the processor 205 via a connection 219. The portable memory interface 206 allows a complementary portable memory device 225 to be coupled to the electronic device 102 to act as a source or destination of data or to supplement the internal storage module 209. Examples of such interfaces permit coupling with portable memory devices such as Universal Serial Bus (USB) memory devices, Secure Digital (SD) cards, Personal Computer Memory Card international Association (PCMIA) cards, optical disks and magnetic disks.

The electronic device 102 also has a communications interface 208 to permit coupling of the electronic device 102 to a computer, a communications network 220, or an external output device (not shown) via a connection 221. The connection 221 may be wired or wireless. For example, the connection 221 may he radio frequency or optical. An example of a wired connection includes Ethernet. Further, an example of wireless connection includes Bluetooth™ type local, interconnection, Wi-Fi (including protocols based on the standards of the IEEE 802.11 family), Infrared Data Association (IrDA) and the like.

As described previously, the electronic device 102 is configured to perform some special functions. The embedded controller 202, possibly in conjunction with further special function components 210, is provided to perform these special functions. For example, where the electronic device 102 is a device for identifying muscles/nerves, and treating neuro-muscular disorders, the special function components 210 may include a current source, an electromyographic processing unit, and a switching unit The special function components 210 are connected to the embedded controller 202.

The methods described with reference to FIG. 4 may be implemented using the embedded controller 202, where the processes may be implemented as one or more software application programs 233 executable within the embedded controller 202. The electronic device 102 of FIG. 2A implements the described methods. In particular, with reference to FIG. 2B, the steps of the described methods are effected by instructions in the software 233 that are carried out within the controller 202. The software instructions may be formed as one or more code modules, each for performing one or more particular tasks. The software may also be divided into two separate parts, in which a first part and the corresponding code modules performs the described methods and a second part and the corresponding code modules manage a user interface between the first part and the operator.

The software 233 of the embedded controller 202 is typically stored in the non-volatile ROM 260 of the internal storage module 209. The software 233 stored in the ROM 260 can be updated when required from a computer readable medium. The software 233 can be loaded into and executed by the processor 205. In some instances, the processor 205 may execute software instructions that are located in the RAM 270. Software instructions may be loaded into the RAM 270 by the processor 205 initiating a copy of one or more code modules from ROM 260 into RAM 270. Alternatively, the software instructions of one or more code modules may be pre-installed in a non-volatile region of RAM 270 by a manufacturer. After one or more code modules have been located in RAM 270, the processor 205 may execute software instructions of the one or more code modules.

The application program 233 is typically pre-installed and stored in the ROM 260 by a manufacturer, prior to distribution of the electronic, device 102. However, in some instances, the application programs 233 may be supplied to the user encoded on one or more CD-ROM (not shown) and read via the portable memory interface 206 of FIG. 2A prior to storage in the internal storage module 209 or in the portable memory 225. In another alternative, the software application program 233 may be read by the processor 205 from the network 220, or loaded into the controller 202 or the portable storage medium 225 from other computer readable media. Computer readable storage media refers to any non-transitory tangible storage medium that participates in providing instructions and/or data to the controller 202 for execution and/or processing. Examples of such storage media include floppy disks, magnetic tape, CD-ROM, a hard disk drive, a ROM or integrated circuit, USB memory, a magneto-optical disk, flash memory, or a computer readable card such as a PCMCIA card and the like, whether or not such devices are internal or external of the electronic device 102. Examples of transitory or non-tangible computer readable transmission media that may also participate in the provision of software, application programs, instructions and/or data to the electronic device 102 include radio or infra-red transmission channels as well as a network connection to another computer or networked device, and the Internet or Intranets including e-mail transmissions and information recorded on Websites and the like. A computer readable medium having such software or computer program recorded on it is a computer program product.

The second part of the application programs 233 and the corresponding code modules mentioned above may be executed to implement one or more graphical user interfaces (GUIs) to be rendered or otherwise represented upon the display 214 of FIG. 2A. Through manipulation of the user input device 112 (e.g., the triggers, switches, buttons), a physician and the application programs 233 may manipulate the interface in a functionally adaptable manner to provide controlling commands and/or input to the applications associated with the GUI(s). Other forms of functionally adaptable user interfaces 112 may also be implemented, such as an audio interface utilizing speech prompts output via loudspeakers (not illustrated) and user voice commands input via the microphone (not illustrated).

FIG. 2B illustrates in detail the embedded controller 202 having the processor 205 for executing the application programs 233 and the internal storage 209. The internal storage 209 comprises read only memory (ROM) 260 and random access memory (RAM) 270. The processor 205 is able to execute the application programs 233 stored in one or both of the connected memories 260 and 270. When the electronic device 102 is initially powered up, a system program resident in the ROM 260 is executed. The application program 233 permanently stored in the ROM 260 is sometimes referred to as “firmware”. Execution of the firmware by the processor 205 may fulfil various functions, including processor management, memory management, device management, storage management and user interface.

The processor 205 typically includes a number of functional modules including a control unit (CU) 251, an arithmetic logic unit (ALU) 252 and a local or internal memory comprising a set of registers 254 which typically contain atomic data elements 256, 257, along with internal buffer or cache memory 255. One or more internal buses 259 interconnect these functional modules. The processor 205 typically also has one or more interfaces 258 for communicating with external devices via system bus 281, using a connection 261.

The application program 233 includes a sequence of instructions 262 through 263 that may include conditional branch and loop instructions. The program 233 may also include data, which is used in execution of the program 233. This data may be stored as part of the instruction or in a separate location 264 within the ROM 260 or RAM 270.

In general, the processor 205 is given a set of instructions, which are executed therein. This set of instructions may be organised into blocks, which perform specific tasks or handle specific events that occur in the electronic device 102. Typically, the application program 233 waits for events and subsequently executes the block of code associated with that event. Events may be triggered in response to input from a user, via the user input devices 112 of FIG. 2A, as detected by the processor 205. Events may also he triggered in response to other sensors and interfaces in the electronic device 102.

The execution of a set of the instructions may require numeric variables to be read and modified. Such numeric variables are stored in the RAM 270. The disclosed method uses input variables 271 that are stored in known locations 272, 273 in the memory 270. The input variables 271 are processed to produce output variables 277 that are stored in known locations 278, 279 in the memory 270. Intermediate variables 274 may be stored in additional memory locations in locations 275, 276 of the memory 270. Alternatively, some intermediate variables may only exist in the registers 254 of the processor 205.

The execution of a sequence of instructions is achieved in the processor 205 by repeated application of a fetch-execute cycle. The control unit 251 of the processor 205 maintains a register called the program counter, which contains the address in ROM 260 or RAM 270 of the next instruction to be executed. At the start of the fetch execute cycle, the contents of the memory address indexed by the program counter is loaded into the control unit 251. The instruction thus loaded controls the subsequent operation of the processor 205, causing for example, data to be loaded from ROM memory 260 into processor registers 254, the contents of a register to be arithmetically combined with the contents of another register, the contents of a register to be written to the location stored in another register and so on. At the end of the fetch execute cycle the program counter is updated to point to the next instruction in the system program code. Depending on the instruction just executed this may involve incrementing the address contained in the program counter or loading the program counter with a new address in order to achieve a branch operation.

Each step or sub-process in the processes of the methods described below is associated with one or more segments of the application program 233, and is performed by repeated execution of a fetch-execute cycle in the processor 205 or similar programmatic operation of other independent processor blocks in the electronic device 102.

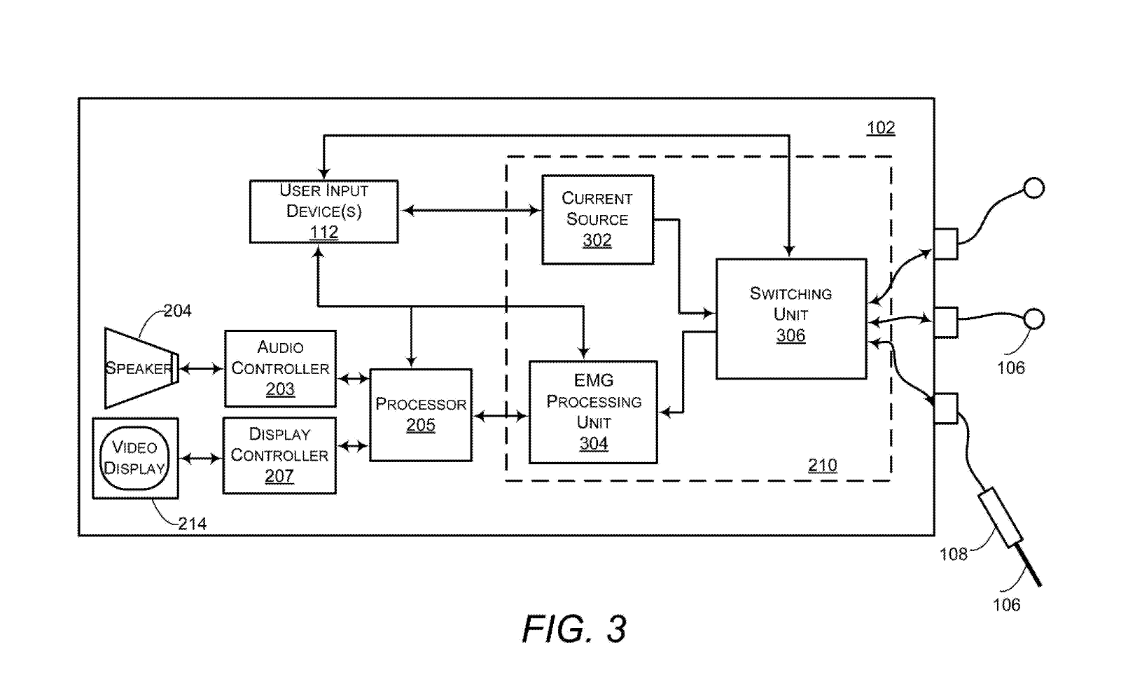

FIG. 3 is a block diagram illustrating some modules of the electronic device 102 in detail. Particularly, FIG. 3 illustrates the special function units 210 of the electronic device 102 in communication with other modules of the electronic device 102. The special functions unit 210 includes a current source 302, an electromyographic processing unit 304, and a switching unit 306. Moreover, the EMG processing unit 304 may be operatively coupled to the display controller 207 and/or the audio controller 203 through the processor 205. In addition, the current source 302, the EMG processing unit 304, and the switching unit 306 may be operatively coupled to the user input devices 112 through the processor 205 to receive one or more operator preferences.

In FIG. 3, the EMG processing unit 304 is illustrated as an independent module, which functions in cooperation with the processor 205. However, it will be understood that in other arrangements, the EMG processing unit 304 may be incorporated within the processor 205 and the functionality of the EMG processing unit 304 may be performed by the processor 205.

The current source 302 may be configured to generate one or more electrical stimulus and transmit these stimuli to the switching unit 306. The stimuli may be constant current, short duration, direct current (DC) stimuli. Further, the current source 302 may generate the electrical stimuli based on configurable parameters such as amplitude, duration, and/or frequency of the electrical stimuli. In one arrangement, the operator may supply the values of the configurable parameters using the input devices 112. As mentioned previously, the input devices 112 may include buttons, switches, toggles, joysticks, or touchscreen interfaces that allow the operator to supply instructions. Depending on the position of the electrodes 106, and the condition of the muscles being stimulated, the operator may increase or decrease the amplitude, duration, or frequency of the generated electrical stimuli. Further, the operator may be allowed to switch “on” or switch “off” the current source 302 as and when required.

Alternatively, the processor 205 may be programmed to supply the values of the parameters associated with the electrical stimuli. To that end, the processor 205 may receive inputs from the EMG processing unit 304 and based on the recorded output of that unit, the processor 205 may be configured to increase or decrease the intensity, duration and/or frequency of the electrical stimuli. For instance, if the processor 205 determines that for a particular amplitude of the electrical stimuli, the patient's muscles twitch excessively, the processor 205 may be configured to reduce the amplitude and/or frequency of the electrical stimuli.

The EMG processing unit 304 may be configured to receive EMG signals from the switching unit 306 and process the EMG signals to produce a corresponding output signal. Further, depending on the type of output required (i.e., audio or visual), the EMG processing unit 304 may be configured to generate output signals such as charts, graphs, or audio streams corresponding to the received EMG signal. In one arrangement, the EMG processing unit 304 may be coupled to the audio controller 203. In this case, the EMG processing unit 304 may be configured to process the EMG signal to produce one or more corresponding audio streams. In other arrangements, the audio controller 203 may be (fully or partly) incorporated within the processor 205 and the functionality of the audio controller 203 may be performed by the processor 205.

Alternatively, the EMG processing unit 304 may be coupled to the display controller 207. In such a case, the EMG processing unit 304 may be configured to generate a visual signal corresponding to the EMG signal. Further, in some arrangements, the display controller 207 may be (fully or partly) incorporated within the processor 205 and the functionality of the display controller 207 may be performed by the processor 205.

In another arrangement, the EMG processing unit 304 may be coupled to both the audio controller and the display controller. In this case, the operator may manually select the desired output signal through use of the user input devices 112. Alternatively, the processor 205 may automatically select the desired output signal based on one or more pre-programmed instructions 233.

An operator may view the output signals on the video display 214 or listen to the output signals on the speaker 204 to identify the muscle being stimulated and the condition of that muscle. Moreover, the generated output signals may be stored in the internal storage 209 or the portable storage medium 225. Accordingly, an operator may interpret the output signals at a later time by retrieving the stored output signals.

Furthermore, in some instances, while transmitting the EMG signal to the EMU processing unit 304, the switching unit 306 may halt the transmission periodically. During this periodic interruption in the EMG signal, a gap is created in the output signal. In one arrangement, the EMG processing unit 304 is configured to fill the created gaps using one or more known techniques. For instance, the EMG processing unit 304 may be configured to interpolate the EMG signal in the corresponding output signal. Any interpolation technique, such as linear or non-linear interpolation, may be utilized without departing from the scope of the present disclosure.

Alternatively, the value of the EMG signal immediately prior to or immediately after the interruption is held for the duration of the interruption. If such a value holding technique is employed, the EMG processing unit 304 may be configured to delay the output signal until the value immediately succeeding the gap is received.

The switching unit 306 is configured to receive electrical stimuli from the current source 302 and deliver the stimuli to the electrodes 106. Also, the switching unit 306 is configured to receive the EMG signal from the electrodes 106 and transmit the EMG signal to the EMG processing unit 304.

The switching unit 306 may operate in three different operation modes. The modes include stimulation, EMG, and simultaneous stimulation and EMG. Controls for selecting the operation mode may be available on the user input devices 112, allowing an operator to switch between modes when desired. Alternatively, the processor 205 may be programmed to automatically select an appropriate mode during operation of the electronic device 102. Moreover, the processor 205 may be configurable so that an operator may input the duration of each mode and the order of the modes to be implemented during operation.

In the stimulation mode, the switching unit 306 may be operatively coupled to the current source 302 and disconnected from the EMG processing unit 304. Further, the operation of the switching unit 306 may include receiving electrical stimuli from the current source 302 and delivering these stimuli to the electrodes 106.

In the EMG mode, the switching unit 306 may be operatively coupled to the EMG processing unit 304 and disconnected from the current source 302. In this configuration, the switching mode may receive the EMG signal from the electrodes 106 and transmit the signal to the EMG processing unit 304.

In the simultaneous stimulation and EMG mode, the switching unit 306 may be operatively coupled to both the current source 302 and the EMG processing unit 304 in a time-domain multiplexed fashion. Accordingly, the switching unit 306 may be configured to receive electrical stimuli from the current source 302, transmit the electrical stimuli to the electrodes 106, receive the EMG signal from the electrodes 106 and transmit the EMG signal to the EMG processing unit 304 in a multiplexed fashion. Moreover, the timing of the multiplexed operation may be determined based on the parameters associated with the electrical stimuli. Accordingly, for the time duration of an electrical, stimulus, the switching unit 306 may be coupled to the current source 302 to receive one electrical stimulus. Subsequently, until the subsequent electrical stimulus is due, the switching unit 306 may be operatively coupled to the EMG processing unit 304. This switching may proceed until the operation mode is changed or the electronic device 102 is switched off.

Moreover, it will be understood that the timing of the time-domain multiplexing changes in correspondence with any changes in the parameters of the electrical stimuli. For instance, for a stimulus duration and frequency of 1 millisecond and 5 Hz, respectively, the switching unit 306 switches between the current source 302 and the EMG processing unit five (5) times in a second. Moreover, the switching unit 306 receives an electrical stimulus from the current source 302 for 1 ms, transmits the electrical stimulus to the electrodes 106, and then switches operation to the EMG processing unit 304, such that switching unit 306 receives the EMG signal from the electrodes 106 and delivers this signal to the EMG processing unit 304 for 199 ms. Subsequently, the switching unit 306 switches back to receive an electrical stimulus from the current source 302 for the next 1 ms before switching back to receive the EMG signal from the electrode for the next 199 ms. The process continues until the electrical stimulus parameters are changed or the operation mode is changed.

Moreover, if during the simultaneous stimulation and EMG mode, the electrical stimulus parameters are altered, the switching unit 306 is configured to correspondingly vary the switching pattern being executed without stopping. For instance, if the operator changes the duration and frequency of the electrical pulses from 1 ms and 5 Hz to 2 ms and 2 Hz, respectively, the multiplexing pattern of the switching unit 302 varies accordingly. In such an example, the switching unit 306 may operate the current source 302 for 2 ms, then switch to the EMG processing unit 304 and operate the EMG processing unit for 498 ms before switching back to the current source 302 and repeating this time-domain multiplexing until the electrical stimulus parameters are changed, the operation mode is changed, or the electronic device 102 is switched off.

In order to switch between the current source 302 and the EMG processing unit 304 multiple times in less than a second, the switching unit 306 includes an array of fast switching solid-state devices such as transistors, relays, and switches. The transistors, relays, and switches may be configured to switch operation between the current source 302 and electrodes 106 or between the electrodes 106 and the EMG processing unit 304 based on the selected operation mode.

FIGS. 4A, 4B, 4C and 4D show graphical representations of the three modes of operation of the electronic device 102 and the output signal generated by the EMG processing unit 304 for the simultaneous stimulation and EMG mode. In FIGS. 4A-4D, the X-axis represents time while the Y-axis represents the amplitude of the signal. FIG. 4A illustrates the stimulation mode 402. As depicted, in the stimulation mode 402, electrical stimuli 403 of specific amplitude 404, duration 405 and frequency 406 are transmitted from the current source 302 to the electrodes 106.

FIG. 4B generally illustrates the EMG mode 408. Here the graphical representation illustrates an EMG signal 410 processed by the EMG processing unit 304 and displayed on the display 214. In the EMG mode, as described previously, the switching unit 306 couples the EMG processing unit 304 to the electrodes 106 and delivers EMG signals from the electrodes 106 to the EMG processing unit 304.

FIG. 4C generally illustrates the simultaneous stimulation and EMG mode 412. As depicted, in this mode, electrical stimuli 414 are interspersed with an EMG output signal 416. Moreover, as previously described, the switching unit 306 switches between the current source 302 and the EMG processing unit 304 based on the parameters of the electrical stimuli.

When the switching mode electrically couples the current source 302 to the electrodes 106, the EMG processing unit 304 is disconnected and the electrodes 106 do not detect any EMG pulses. Accordingly, gaps are introduced in the EMG signal output each time the electrical stimuli are delivered to the electrodes 106. The EMG processing unit 304 may be configured to fill the introduced gaps either by holding the value of the EMG pulse received immediately prior to disconnection until a subsequent EMG pulse is processed. Alternatively, the EMG processing unit 304 may interpolate the values between the value of the EMG signal received before disconnection and the value of the EMG signal received upon reconnection. Any known interpolation technique may be utilized without departing from the scope of the present disclosure. For instance, linear or non-linear interpolation techniques may be utilized.

FIG. 4D generally depicts an output signal 420 displayed on the video display 214 for the simultaneous stimulation and EMG mode. As depicted, the gaps are filled by interpolation (as shown in gaps 422, 424, and 426), by holding the previous signal value (as shown in gap 428), or by holding the value of the signal immediately after the gap (as shown in gap 430).

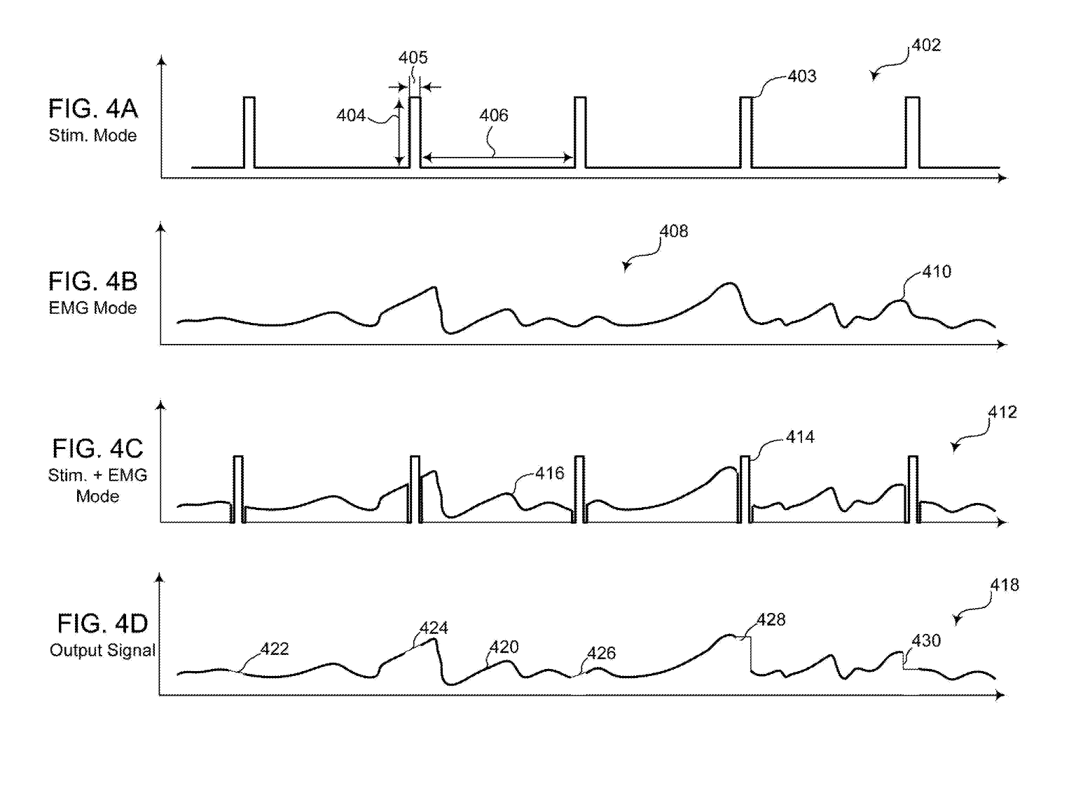

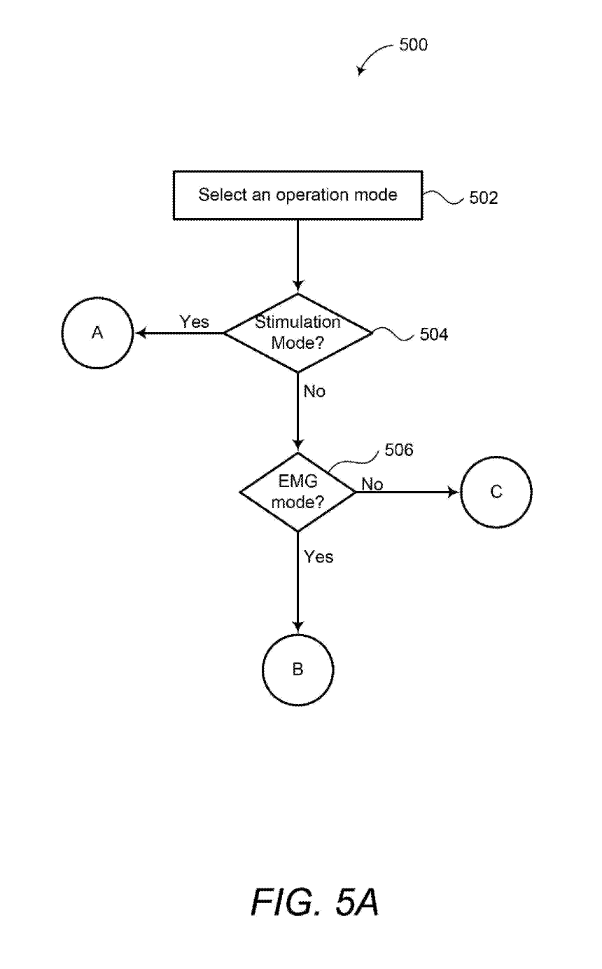

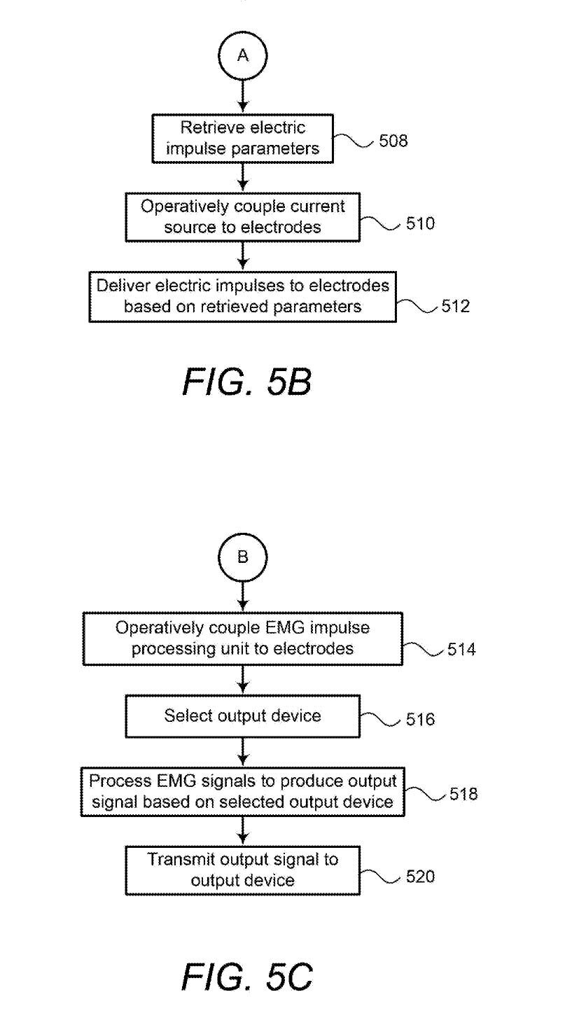

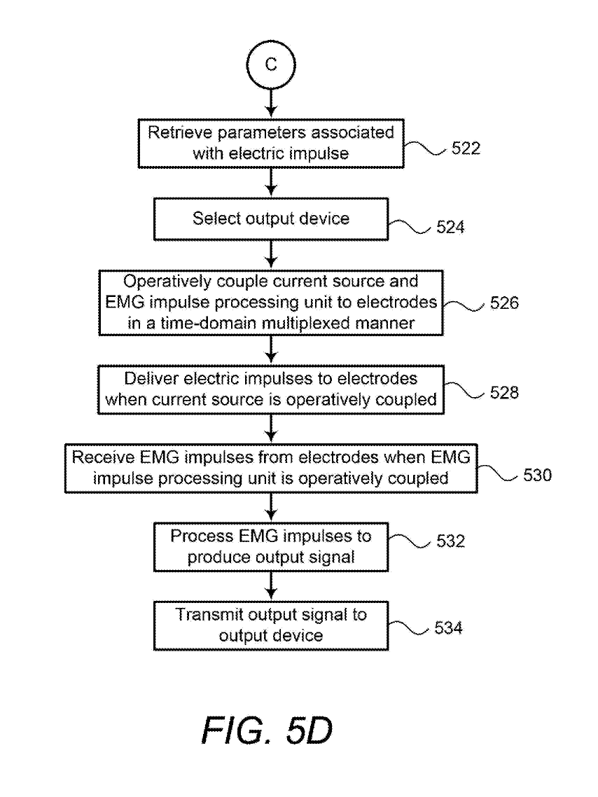

FIGS. 5A-5D collectively form a flowchart illustrating a method 500 for treating a neuromuscular disorder. The method 500 will be described with reference to FIGS. 1-4. In one arrangement, the steps of the method 500 may be implemented as one or more software code modules of the software application program 233 resident in the ROM 260 and being controlled in execution by the processor 205. Moreover, one or more method steps may be deleted, added, or reordered without departing from the scope of the present disclosure. The method 500 begins at step 502, where an operation mode is selected. As described previously, an operator may select the operation mode using one or more user input devices 112 present on the electronic device 102. As described previously, the electronic device 102 may include three operation modes—stimulation, EMG, and simultaneous stimulation and EMG mode. Further, the mode may be automatically selected under execution of the processor 205 or manually selected by an operator using the input devices 112. The selected operation mode is determined at step 502 using the processor 205.

At step 504, a determination is made whether the stimulation mode is selected. If the stimulation mode is selected (yes path from step 504), the method 500 proceeds to process A. Else, the method 500 proceeds to step 506, where a determination is made whether the EMG mode is selected. At step 506, if the EMG mode is selected, the method 500 proceeds to process B. Else, the method 500 proceeds to process 522 (generally indicated by “C”).

FIG. 5B depicts process A—the stimulation mode. The process A begins at step 508, where one or more electrical parameters associated with electrical stimuli are retrieved. As described previously, the parameters may be automatically selected by the processor 205 from the internal storage module 209. Alternatively, the parameters may be manually selected by an operator via the user input devices 112. The parameters include amplitude 404, duration 405, and the frequency 406 of the electrical stimuli.

Once the parameters associated with the electrical stimuli are retrieved, the current source 302 is operatively coupled to the electrodes 106. In one arrangement, the switching unit 306 may operatively couple the current source 302 to the electrodes 106. Subsequently, the electrical stimuli may be delivered to the electrodes based on the retrieved parameters, at step 512. To that end, the current source 302 may be configured to generate the electrical stimuli based on the retrieved parameters and the switching unit 306 may be configured to deliver the generated electrical stimuli to the electrodes 106. The electrodes 106 may be placed on the skin of the patient 104 or in a percutaneous layer. Accordingly, the electrical stimuli may be delivered to a specific location in the patient's body.

FIG. 5C depicts process B—the EMG mode. The process B begins at step 514, where the EMG processing unit 304 is operatively coupled to the electrodes 106. In one arrangement, the switching unit 306 is configured to operatively couple the EMG processing unit 304 to the electrodes 106.

Subsequently, an output device is selected (optional step). In some cases, the electronic device 102 may include one output device (such as a video display or a speaker). In such a case, output device selection step 516 may be skipped. In arrangements where the electronic device 102 includes two or more output devices, step 516 is implemented. Accordingly, at step 516, an operator may manually select an output device from a list of output devices available to the electronic device 102 or the processor 205 may automatically select the output device. It will be understood that the output devices may be internal or external to the electronic device 102 and the output devices may include video displays, speakers, or a combination thereof.

Next, at step 518, the EMG signal is processed to form an output signal based on the selected output device. To that end, the switching unit 306 delivers the EMG signal detected at the specific muscle/nerve site to the EMG processing unit 304. In turn, the EMG processing unit 304 may process the signal to generate the output signal. Processing may include one or more of filtering the EMG signal, amplifying a portion of the EMG signal, attenuating a portion of the EMG signal, converting the EMG signal into a corresponding chart, graph, or audio stream.

Once the EMG signal is processed into an output signal, the output signal is transmitted to the output device at step 520. To this end, the EMG processing unit 304 may transmit the output signal to an output device controller and the controller in turn may further process the output signal before transmitting the processed output signal to the output device.

FIG. 5D depicts process C—simultaneous stimulation and EMG mode. The process C begins at step 522, where the parameters associated with electrical stimuli are retrieved. Next, the output device is selected (at step 524). Subsequently, at step 526, the current source 302 and the EMG processing unit 304 are coupled to the electrodes 106 in a time-domain multiplexed fashion. To that end, the current source 302 and the EMG processing unit 304 are coupled to the switching unit 306, which in turn is coupled to the electrodes. The switching unit 306 is configured to switch operation between the current source 302 and electrodes 106 and the EMG processing unit 304 and the electrodes 106 in the time-domain multiplexing fashion. Furthermore, the multiplexing timing of the switching unit 206 are configurable based on the retrieved parameters associated with the electrical stimuli.

At step 528, one or more electrical stimuli are delivered from the current source 302 to the electrodes 106. When the current source 302 is operatively coupled to the electrodes 106, electrical stimuli is delivered to the patient 104 to stimulate muscle contraction.

At step 530, the EMG signal is received from the electrodes 106. When the EMG processing unit 304 is coupled to the electrodes 106, EMG signal is received from the contracting muscles of the patient 104 through the electrodes 106 and the switching unit 306.

The EMG signal received from the electrodes 106 at the previous step is processed at step 528. Particularly, the EMG signal is processed in the EMG processing unit 304 to generate an output signal. Moreover, because the received EMG signal is interspersed with transmitted electrical stimuli, gaps may be present in the received EMG signal at instances when the EMG processing unit 304 is disconnected and the current source 302 is connected to the electrodes 106. To fill the gaps, the EMG processing unit 304 may interpolate the values immediately prior to the disconnection and immediately after the reconnection. Alternatively, the processing unit 304 may hold the value of the EMG signal before disconnection. Finally at step 532, the processed output signal is transmitted to the selected output device.

Furthermore, an operator may evaluate the output signals and determine whether the correct muscle/nerve is identified. If the operator determines that an incorrect muscle/nerve is being stimulated, the operator may reposition the electrodes 106 and repeat the process, until the correct muscles/nerves are identified. Moreover, in case the device 102 is utilized to inject a paralysing agent, upon determination of the correct muscle/nerve, the operator may inject the paralysing agent to the target muscle/nerve through the syringe 108 to treat the underlying disorder.

Devices and methods described in the present disclosure may substantially simultaneously stimulate muscles/nerves of a patient and receive EMG signals associated with the muscles/nerves. Moreover, in the simultaneous stimulation and EMG operation mode, the devices and methods automatically switch between stimulation and EMG by employing a time-domain multiplexing switching unit.

In conventional devices, a physician had to manually switch between the stimulation and EMG modes, thereby potentially moving the device during the procedure. Because of such movement, the needle electrode connected to the conventional devices often shifted during the procedure, which potentially led to injecting the paralysing agent in an incorrect site. In the present disclosure, however, as the switching is automatically performed by the electronic device, a physician does not need to move his/her hands during the procedure, thereby increasing the accuracy with which the injection site is located. Moreover, as the stimulation and EMG modes are carried out substantially simultaneously, the electronic device of the present disclosure may aid physicians in locating the injection site quicker than traditional devices.

The foregoing describes only some embodiments of the present disclosure, and modifications and/or changes can be made thereto without departing from the scope and spirit of the present disclosure, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

1. An electronic device for treating neuromuscular disorders, the electronic device comprising:
a current source configured to generate one or more electrical stimuli based on one or more selected parameters associated with the one or more electrical stimuli;
an electromyographic (EMG) processing unit configured to process a received EMG signal; and
a switching unit operatively coupled to the current source and the EMG processing unit
wherein in a simultaneous EMG and stimulation mode, the switching unit is operatively coupled to the current source and the EMG processing unit in a time-domain multiplexed manner such that the switching unit delivers the one or more electrical stimuli to a patient and receives the EMG signal from the patient substantially simultaneously.

2. The electronic device of claim 1, wherein the switching unit is configured to operate in an EMG mode, a stimulation mode, and a the simultaneous EMG and stimulation mode.

3. The electronic device of claim 2, wherein:
in the EMG mode, the switching unit is operatively coupled to the EMG processing unit and configured to receive the EMG signal from a patient; and
in the stimulation mode, the switching unit is operatively coupled to the current source and configured to transmit the one or more electrical stimuli to the patient.

4. The electronic device of claim 1, wherein the switching unit comprises one or more switching array configured to perform the time-domain multiplexing.

5. The electronic device of claim 1, wherein the switching unit is configured to stop receiving the EMG signal when the one or more electrical stimuli are delivered to the patient.

6. The electronic device of claim 1, wherein the EMG processing unit is configured to interpolate the received EMG signal when the one or more electrical stimuli are delivered to the patient.

7. The electronic device of claim 1 further comprising an output unit configured to generate an output signal corresponding to the processed EMG signal.

8. The electronic device of claim 7, wherein the output unit is at least one of a video display or a speaker.

9. The electronic device of claim 7, wherein the system comprising one or more user input devices configured enable an operator to:
select a predetermined parameter associated with the one or more electrical stimuli;
control operation of the output unit; and
select the operation mode of the system.

10. The electronic device of claim 1, further comprising one or more electrodes coupled to the switching unit.

11. An electronic device for treating a neuro-muscular condition, the electronic device comprising:
a current source configured to generate one or more electrical stimuli based on a predetermined parameter;
an electromyographic (EMG) processing unit configured to receive and process an EMG signal;
a switching unit operatively coupled to the current source and the EMG processing unit in a time-domain multiplexed manner, the switching unit configured to deliver the one or more electrical stimuli from the current source to a patient and transmit the EMG signal from the patient to the EMG processing unit, substantially simultaneously; and
an output unit configured to generate an output signal corresponding to the processed EMG signal.

12. The electronic device of claim 11, wherein the switching unit comprises one or more switching array configured to perform the time-domain multiplexing.

13. The electronic device of claim 12, wherein the switching unit is configured to disconnect the EMG processing unit when the current source is operatively coupled to the switching unit and disconnect the current source when the EMG processing unit is operatively coupled to the switching unit.

14. The electronic device of claim 13, wherein the EMG processing unit is configured to interpolate the EMG signal when the current source is operatively coupled to the switching unit and the EMG processing unit is disconnected.

15. The electronic device of claim 11, wherein the output unit is at least one of a display device or an audio device.

16. The electronic device of claim 11, wherein the system comprising one or more controls configured to:
select a predetermined parameter associated with the one or more electrical stimuli;
control operation of the output unit; and
control the operation mode of the system.

17. The electronic device of claim 1, further comprising one or more electrodes coupled to the switching unit, wherein the one or more electrodes are configured to receive the EMG signal from the patient and transmit the one or more electrical stimuli to the patient.

18. A method for treating a neuromuscular disorder, the method comprising:
retrieving one or more parameters associated with electrical stimuli;
operatively coupling a current source and an EMG processing unit to a patient in a time domain multiplexed manner, wherein the timing of the multiplexing operation is based on the one or more retrieved parameters of the electrical stimuli;
delivering one or more electrical stimuli from the current source to a patient when the current source is operatively coupled to the patient;
receiving an EMG signal from the patient, when the EMG processing unit is operatively coupled to the patient;
processing the received EMG signal to generate an output signal; and
transmitting the output signal to an output device.

19. The method of claim 18, further comprising interpolating the received EMG signal when the current source is operatively coupled to the patient.

20. The method of claim 18, further comprising holding a value of the EMG signal when the current source is operatively coupled to the patient.
